(19) United States
(12) Patent Application Publication
(10) Pub. No.: US 2017/0014041 A1
(43) Pub. Date: Jan. 19, 2017

(54) SIMPLE HEART RATE MONITOR SHOWING MINUTES BELOW, IN, AND ABOVE A HEART RATE ZONE

(71) Applicant: KONINKLIJKE PHILIPS N.V., EINDHOVEN (NL)

(72) Inventors: Wilhelmus Johannes Joseph Stut, Eindhoven (NL); Francesco Sartor, Eindhoven (NL); Michael Heesemans, Eindhoven (NL); Gerhard Spekowius, Straelen (DE); Jennifer Caffarel, Eindhoven (NL)

(21) Appl. No.: 15/279,614

(22) Filed: Sep. 29, 2016

Related U.S. Application Data
(63) Continuation of application No. PCT/EP2015/056166, filed on Mar. 24, 2015.

(30) Foreign Application Priority Data
Apr. 1, 2014 (EP) 14163066.5

(51) Int. Cl. A61B 5/024; A61B 5/00

(52) U.S. Cl. CPC A61B 5/02438; A61B 5/6824; A61B 5/6815; A61B 5/6823; A61B 5/6831; A61B 5/7455; A61B 5/7282

(57) ABSTRACT

Various embodiments relate to a system and method for monitoring the heart rate of a user. In particular various embodiments relate to a system comprising an input unit, a time measurement unit, a heart rate sensor, a processing unit and an output unit. The processing unit determines at least one of three different time periods, the first below, the second inside and the third above a single heart rate range defined by the user.

### TECHNICAL FIELD

Various embodiments relate to a system and method for monitoring the heart rate of a user. More particularly, but not exclusively, various embodiments include a system comprising an input unit, a time measurement unit, a heart rate sensor, a processing unit and an output unit. The processing unit determines at least one of three different time periods, the first below, the second inside and the third above a defined single heart rate range.

### BACKGROUND

Physical activity and exercise are essential to improve the condition of people with chronic diseases (like heart failure, COPD, diabetes, high blood pressure, sleep apnea) and related events (such as myocardial infarction). Unfortunately, most people with these conditions are not offered appropriate programs or do not reach the recommended level of physical activity and exercise.

Several guidelines for physical activity and exercise have been published. The American College of Sports Medicine has published guidelines for exercise management of chronically ill patients. The European Society of Cardiology has published guidelines for physical activity and exercise for cardiac patients.

These guidelines give a recommendation regarding the number of days per week, the length of the sessions, and the intensity (typically a heart rate (HR) zone). For example, for heart failure patients, the ESC recommends exercise for 3-5 days per week, 20-60 minutes per session and heart rate between 70-85% of the maximum heart rate (HRmax). The recommendation for physical activity encompasses its performance on most days of the week and for 30-60 minutes with moderate intensity (i.e. HR below 70% of HRmax).

Furthermore, if patients are recommended both exercise and physical activity, the amount of physical activity on exercise days may be lower than on non-exercise days. For example, for a particular patient with HRmax=120, the prescription could be as follows: exercise for 3 days per week, 20 minutes per session and HR between 84 and 102. Physical activity for all days of the week, for exercise days 10 minutes and other days 40 minutes with moderate intensity (i.e. HR below 84).

Due to the growing number of people that suffer the above mentioned conditions or live an inactive life, many physical activity promotion products and services have been developed over the last decades, both for research and commercial objectives. Said physical activity promotion products in most cases try to calculate or estimate the heart rate in order to display the person's pulse during a physical activity. At present, the most successful devices that measure the heart rate for athletes make use of a chest belt. For example, an electrocardiographic (ECG) device detects electrical activity that corresponds to muscle excitation of the heart. A pulse oximeter (or photoplethysmogram (PPG)) determines changes in light absorption in which the changes are indicative of blood flow through an anatomical part, such as a finger. A phonocardiograph (PCG) is another type of monitoring system that detects sounds caused by the closing of heart valves. All of the above monitoring systems have in common that they may be used to detect oscillating waveforms that correspond to heart beats.

Because more and more people are aware of the power of monitoring heart rate for their health, and most people try to avoid wearing such a kind of chest strap due to its uncomfortableness, the paradigm of measuring the heart rate slowly changes from high resolution, low comfort and difficult handling to medium or even low resolution but higher wearing comfort and considerably facilitated handling. This is achieved, for example, through optical heart rate monitors, which may be attached to different parts of the body, e.g. also to the wrist of the user.

### SUMMARY

Various embodiments provide improvements to heart rate monitors. In particular, various embodiments provide a simpler and more efficient heart rate monitor. Various embodiments include the provision of a heart rate monitor which is easy to operate. Still another objective resides in the provision of a heart rate monitor which may be easily operated during use.

Various embodiments relate to a system for monitoring heart rate of a user is presented that comprises an input unit for defining a single heart rate range, a time measurement unit, a heart rate sensor for measuring the heart rate of the user, and a processing unit for determining a first time period T₁ the measured heart rate is below the defined single heart rate range, a second time period T₂ the measured heart rate is inside the defined single heart rate range and a third time period T₃ the measured heart rate is above the defined single heart rate range, and an output unit for indicating at least one of the first, second and third time periods.

Various embodiments relate to a method for monitoring the heart rate of a user is presented that comprises defining a single heart rate range, measuring the heart rate of the user, a first time period T₁ the measured heart rate is below the defined single heart rate range, a second time period T₂ the measured heart rate is inside the defined single heart rate range and a third time period T₃ the measured heart rate is above the defined single heart rate range, and indicating at least one of the first, second and third time periods.

Various embodiments therefore overcome the above-mentioned disadvantages by providing the presented system and method for monitoring the heart rate of a user, wherein essential information about the question if the heart rate is in a defined limit is indicated in a readily comprehensible manner. The presented system and method are characterized by a facilitated definition of a single heart rate zone by either defining upper and lower values of an acceptable heart rate or defining an optimal heart rate value and an acceptable deviation. In addition, the system and method afford counting and indicating the at least one of the time period below, inside and above the single heart rate range as defined. Advantageously, the system has no button to start and/or stop a training session.

The presented system and method for monitoring the heart rate allows the user to define one or a single heart rate zone (e.g. from 84 to 102 beats per minute) to easily determine and indicate the time period below, in, and above the heart rate zone. The present system and method thereby permit the user to specifically address his/her needs by choosing either a training program for exercise purposes, wherein the heart rate is in the recommended zone, or for physical activity, wherein the heart rate below the recommended zone.

At least some parts of the system may be accommodated e.g. in a wrist-worn device or in an in-ear or ear-worn device. Such devices are well known in the art and ensure that the heart rate sensor is in contact with the skin of the user. It will be obvious that the present system may be included in a single device or may be in form of several devices remote from each other. For example, the system may be in form of a single device, including input unit, time measurement unit, heart rate sensor processing unit and output unit. The output unit may comprise visual, acoustic and/or haptic devices.

Some parts of the system may be located remote from each other. For example, input unit and output unit may be in form of an app shown for example on the screen of a mobile phone. An “app” as used herein is a piece of software which can run on the internet, computer, mobile phone or any other electronic device. Alternatively, the heart rate sensor may be remote from the input unit, time measurement unit, processing unit and output unit. In addition, there may be more than one output unit, such as two or three output units. This permits not only the user to prosecute the current training/exercise program, but also allows a medical practitioner to follow up and to interfere, if necessary.

An input unit for the system may be any kind of input means perceivable. If the input unit is located together with all other system's components, it may be beneficial that the input unit comprises a single button, such as a mechanical or electronic button. Such a single button may be used to switch on/off the system as well as for defining the single heart rate range. It is conceivable to employ two or more buttons, such as three, four or five buttons. It is also possible to include other control elements such as adjustment wheels or to use instead of a button an adjustment wheel having additional button functionality. It is also possible to use a touchscreen.

The input unit may be also at a remote location. In such a case the input unit may comprise an app usable for a common mobile device, such as a smartphone. This has the advantage that the part of the system or the portable device, i.e. the device comprising the heart rate sensor, has small and compact dimensions and may be easily attached to the user's skin. A remote input unit is also conceivable in case of any remote control unit which may be used for example to adjust the heart rate range and which is then not required anymore. In addition a remote control unit may be in form of any computer system which allows for example a medical practitioner to adjust required values.

The time measurement unit may be a clock-like device which may be also included in the processing unit or form part thereof The time measurement unit is used to determine the heart rate in conjunction with the heart rate sensor and the processing unit permitting calculation/determining of a current heart rate. In addition, the time measurement unit is employed by the processing unit to determine at least one of the first time period the measured heart rate is below the defined single heart rate range, the second time period the measured heart rate is inside the defined single heart rate range and the set time period the measured heart rate is above the defined single heart rate range. The time measurement unit may be alternatively used to indicate the current time on the output unit and/or to give other time-related measures.

The heart rate sensor for measuring the heart rate of the user may be any suitable device known in the art. Examples comprise pulse oximeters or photoplethysmographs (PPG) sensors and phonocardiograph (PCG) sensors which may be readily employed in the systems and methods described herein. The heart rate may be measured continuously or discontinuous.

The processing unit determines at least one of the first time period the measured heart rate is below the defined single heart rate range, the second time period the measured heart rate is inside the defined single heart rate range and the third time period the measured heart rate is above the defined single heart rate range. In other words, the processing unit is adapted for determining one or more of said three time periods. The processing unit may indicate one of the first time period T₁, the second time period T₂ and the third time period T₃. In some embodiments, the output unit indicates sequentially or simultaneously a combination of the first time first time period T₁ and the second time period T₂. Alternatively the output unit may indicate sequentially or simultaneously a combination of the first time first time period T₁ and the third time period T₃. Some embodiments use a sequentially or simultaneous indication of the first time first time period T₁, the second time period T₂ and the third time period T₃. It will be appreciated that also determining of either T₂ alone or a combination of T₁ and T₃ is advantageous as the user may readily gasp the relevant information.

In addition, the processing unit calculates the current heart rate on the basis from the signals derived from the heart rate sensor and the time measurement unit. Control of the processing unit is performed by the input unit, an output is provided by the output unit. In addition, the processing unit may employ signals from other sensors, such as a motion sensor and acceleration sensor. The data of these sensors may be either used for controlling the function of the device and/or providing other indications on the output unit.

The output unit indicates the at least one of the first, second and third time periods characterizing the three time periods in question, namely wherein the measured heart rate is below, inside or above the defined single heart rate range. This permits an immediate and perceivable possibility to the user to grasp the relevant information. The output unit or renderer may be a display, such as touchscreen display used also as input unit.

The output unit for indicating at least one of the first, second and third time periods may be a visual and/or acoustical output unit. Examples for visual output units comprise screens of devices, such as touchscreens of a mobile device or computer, touchscreens or screens of a handheld device, or a simple display in combination with LEDs. Some embodiments utilize a combination of three LEDs with a screen, whereas the color of the LED indicates the measured heart rate below, inside or above the defined single heart rate range and the screen indicates information about the respective time period(s). The period may be either given in seconds, minutes or in form of a common time indication composed of hours, minutes and seconds. An additional advantage of such a simple display resides in the low energy consumption in comparison to a screen.

Examples for acoustic indications comprise acoustic announcements or spoken language such as “10 minutes inside, 2 minutes above, and 2.5 minutes below the defined heart rate range”.

It will be appreciated that the present system and method do not require that the output unit shows the exactly the same time periods previously determined by the processor but may also show less. This holds true in case the processor determines two or three time periods. In this case it is nevertheless conceivable that indication of one or two of these time periods is sufficient and/or desired. For example, the processor determines all three time periods. The user may, however, desire indication of the second time period only. The user may also desire indication of the first time period alone or indication of the first and third time periods. It will be appreciated that the system and method may be adapted likewise.

A memory unit (not shown) may be encompassed to store user inputs as well as training results for showing to the user after finishing of the training.

Various embodiments use a motion sensor; such a sensor may experience few motion artifiacts, especially in case of large and fast movements as these occur during a physical activity like running, cycling or rowing. Large movement artifacts may complicate the heart rate measurement. In order to compensate artifacts, additional motion sensors may be included in a device to measure the occurring motion of the body part and to compensate for the resulting motion artifacts.

The system may also comprise a button configured to enter a perceived exertion of an exercise or an activity. Such a functionality permits the user in case of increased or decrease perceived exertion to adapt the defined single hear rate range in a corresponding manner. In case of increased perceived exertion pressing of said button may shift the defined single heart rate range for e.g. 5%, 10%, 15%, or 20%. Repeated pressing of said button may cause another shift with the same amount. As noted above combinations of visual and acoustic indications are conceivable.

Alternatively, the present system for monitoring heart rate of a user may comprise an input unit configured to define a single heart rate range, a time measurement unit, a heart rate sensor configured to measure the heart rate of the user, a processing unit configured to determine at least one of a first period T₁ the measured heart rate is below the defined single heart rate range, a second period T₂ the measured heart rate is inside the defined single heart rate range and a third time period T₃ the measured heart rate is above the defined single heart rate range, and an output unit configured to indicate the at least one of the first, second and third time periods.

The use of the present system for monitoring the heart rate of a user is encompassed as well.

### BRIEF DESCRIPTION OF THE DRAWINGS

These and other aspects of the principles, methods, and systems disclosed herein will be apparent from and elucidated with reference to the embodiment(s) described hereinafter. In the following drawings

FIG. 1 shows a schematic appliance of a system;

FIG. 2 shows a schematic block diagram illustrating the components of the system according to a first embodiment;

FIG. 3 shows a schematic block diagram illustrating in- and output signals of a processor of the system according to the first embodiment;

FIG. 4 shows a first example of a measured heart rate signal and an indication of the defined single heart rate range;

FIG. 5 shows a second example of a measured heart rate signal including a defined single heart rate range;

FIG. 6 shows a schematic block diagram illustrating the components of the system according to a second embodiment;

FIG. 7 shows a schematic block diagram illustrating in- and output signals of a processor of the system according to the second embodiment; and

FIG. 8 schematically shows an appliance of the system according to a third embodiment.

### DETAILED DESCRIPTION

FIG. 1 schematically shows an appliance of the system which is denoted by reference numeral 10. A person 40 that is in this figure exemplarily shown as a runner, wears the system or portable device 10 for measuring the pulse during his/her physical activity and/or exercise program.

The system 10 is attached to a body part 42, which body part 42 is suitable for measuring the pulse of the person 40, i.e. the body part 42 on which the arterial blood pulse can be easily tracked. As shown in FIG. 1, the portable device may be attached to the wrist of the person 42. The output unit (20) may comprise in this case visual, acoustic and/or haptic feedback devices. However, the portable device may also be attached to any other part 42 of the person 40, e.g. the chest, a leg, around the neck, at or in an ear. It will be appreciated that in this case in may be necessary to employ a remote visual feedback device, such as a display at the wrist. In addition or alternatively, acoustic and/or haptic feedback devices may be still employed in the portable device.

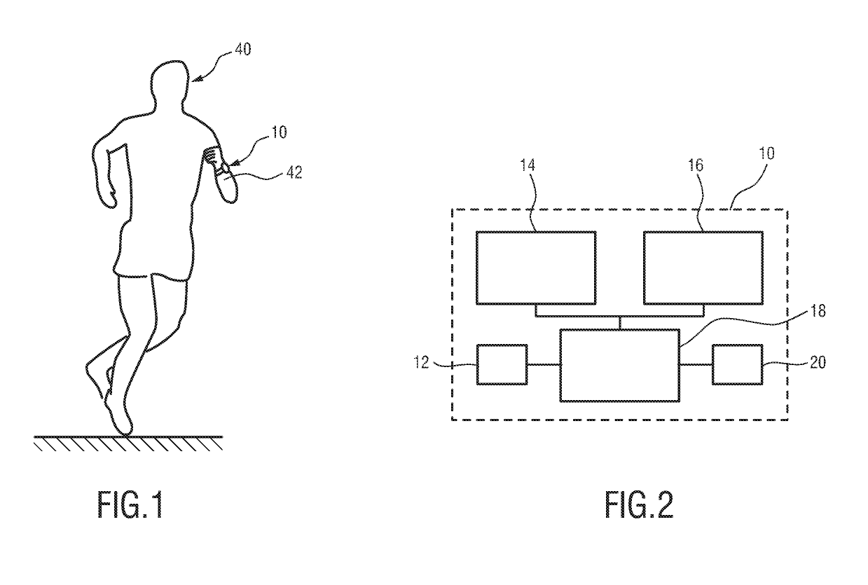

As it is shown in the schematic block diagram of FIG. 2, said system 10 comprises an input unit 12, a time measurement unit 14, a heart rate sensor 16, a processing unit 18, and an output unit 20. Input unit 12, time measurement unit 14, heart rate sensor 16, and output unit 20 are electronically coupled with the processing unit 18. The heart rate sensor 16 may comprise an optical sensor, in particular a photoplethysmography (PPG) sensor, which measures the blood pulse wave of the person 40 over time and generates a heart rate signal 22. In this example, the processing unit 18 determines all three time periods T₁, T₂ and T₃ which are in turn indicated by the output unit 20. It will be clear, that the output unit 18 may alternatively determine only one or two of said time periods which are indicated by the output unit 20.

The photoplethysmography sensor includes a photodetector (not shown) that measures the absorbance of the blood at different wavelengths allowing a determination of the light absorbance changes that are due to the pulsing arterial blood. Such a kind of PPG sensor allows measuring the pulse of the person in a comfortable way.

Alternatively, the heart rate sensor 16 may comprise a chest strap as this is known in the art.

The time measurement unit 14 together with the heart rate sensor 16 generate signals allowing the processing unit 18 to determine the current heart rate 26 and further on the basis of a defined single heart rate range 28, 30 to determine and indicate a first time period T₁ the measured heart rate 26 is below the defined single heart rate range 28, 30, the second time period T₂ the measured heart 26 is inside the defined single heart rate range 28, 30 and a third time period T₃ the measured heart rate 26 is above the defined single heart rate range 28, 30. The term “inside” as used herein is to be understood to comprise the boundary values of the defined single range. Taking for example a determined hear rate range 28, 30 of 100 to 120 beats per minute, T₁ comprises all values smaller than 100 beats per minute, T₂ all values between 100 and 120 beats per minute including 100 and 120 beats per minute, and T₃ all values above 120 beats per minute.

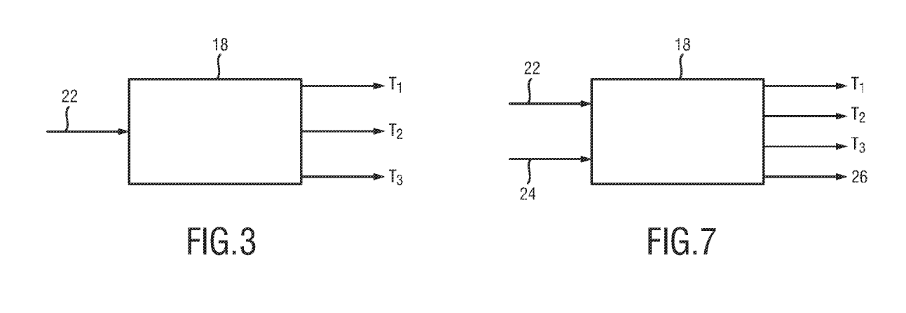

As exemplified in FIG. 3 the heart rate signal 22 is used by the processing unit 18 to output T₁, T₂, and T₃ indicative for the first time period T₁ the measured heart rate is below the defined single heart rate range 28, 30, the second time period T₂ the measured heart rate is inside the defined single heart rate range 28, 30 and the third time period T₂ the measured heart rate is above the defined single heart rate range 28, 30, respectively. The output unit 20 may indicate the at least one of the first, second and third time periods T₁, T₂ and T₃ only. Additionally, the output unit may also indicate the defined single heart rate range 28, 30 and/or a current measured heart rate 26. Example 3 is, however, not limited to processing and indication of three time periods. Processing and indication of one or two time periods is encompassed as well.

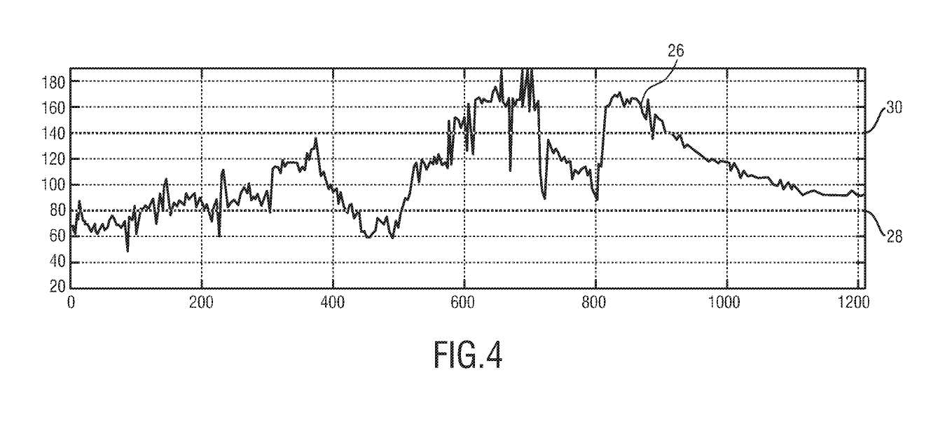

FIG. 4 shows a third example of the measured heart rate 26 of a user indicating upper and lower limits 28, 30 of a defined single heart rate range. The heart rate range 28, 30 is in this example 80 and 140 beats per minute, respectively, as indicated on the y-axis. The x-axis shows the time in seconds.

As may be derived from the example shown in FIG. 4, the measured heart rate 26 is below the lower limit or threshold 28, partially in the time period from zero to approximately 100 seconds, and at a later stage between 580 and 700 seconds above the upper limit or threshold 30. In some embodiments, the presented system is programmed not to indicate a first time period below the defined single heart rate range, as said period may be recognized as a warm-up period which is not relevant for training purposes. The second time period below the lowered limit 28 is, however, relevant and will be later output on the output unit 20 in form of a visual indication showing the time period in minutes or seconds. The summarized time period above the upper limit or threshold 30 is indicated as well. In addition, the relevant training period within the upper and lower limits 30, 28 is shown. In some embodiments the overall time limit of the exercise, i.e. the sum of all three time periods is shown on the display of the device. In addition, complete training time may be given and percentage values of the three time periods with respect to the complete training time. As noted above, this example is not limited to processing and indication of three time periods. Processing and indication of one or two time periods is encompassed as well.

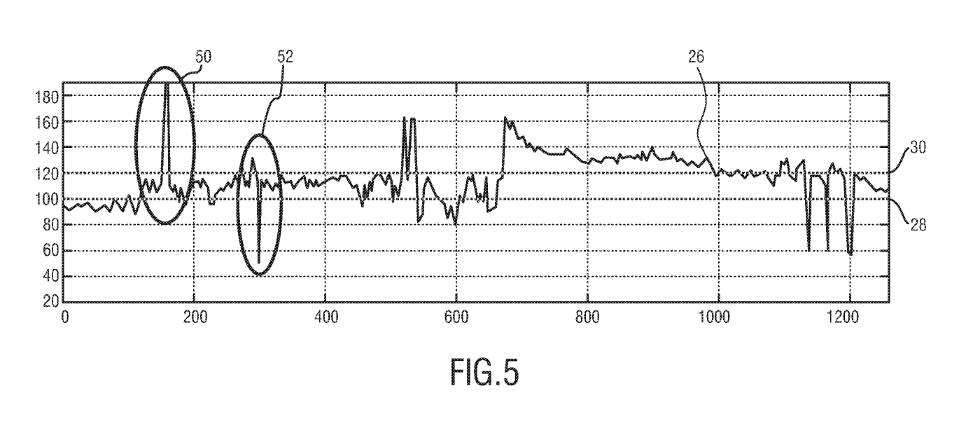

FIG. 5 shows another example of a measured heart rate 26 wherein lower and upper limits 28, 30 are set to 100 and 120 beats per minute. These values may be either individually set by the user, i.e. by input the values 100 and 200 or by indicating upper third value (not shown) and setting a deviation yielding the heart rate range 28, 30 shown in FIG. 5. The system may be adapted to create and indicate an alert as the result of heart rate values considerably deviating from the defined single heart rate range 28, 30. For example an alert may be created upon the first measured heart rate exceeding the defined single heart rate range 28, 30 by far, such as after approximately 155 seconds shown in FIG. 5 and which has been indicated by reference number 50. Other deviations may be considered marginal, such as the deviation after approximately 280 seconds indicated by reference number 52, and will not cause any alert. The user may set threshold values for setting an alert. An alert may be indicated by an acoustic signal sequence or a vibration signal sequence. In some embodiments such signal sequences generated by an acoustic unit (not shown) or vibration unit 32 are defined by the user. The use of a vibration unit 32 may be less disturbing for the user.

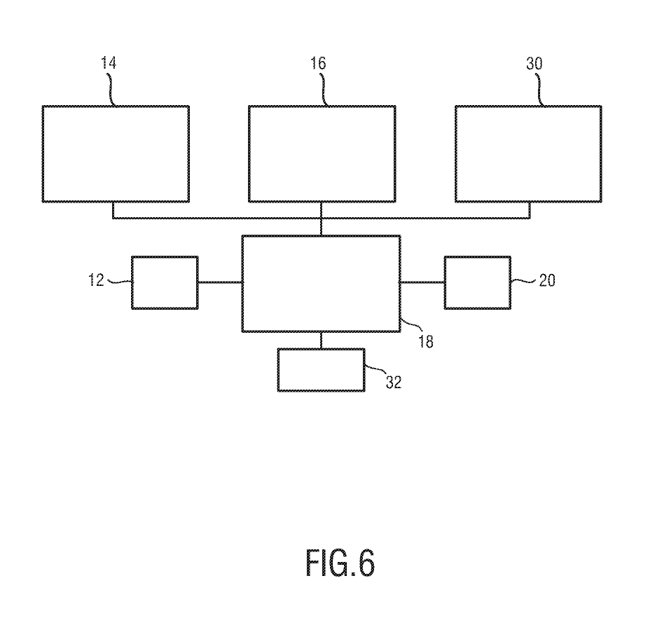

The schematic block diagram of FIG. 6 indicates the components of the system according to another embodiment. The components are input unit 12, time measurement unit 14, heart rate sensor 16, processing unit 18, output unit 20, motion sensor 30, and vibration unit 32. Input unit 12 and output unit 20 may both take use of the same app (not shown) for controlling an indication purposes. The app may comprise a functionality or button for entering a perceived exertion of an exercise or an activity. In case of increased perceived exertion pressing of said button shifts the defined single heart rate range for e.g. 5%, 10%, 15%, or 20%. Repeated pressing of said button may cause another shift with the same amount.

The motion sensor 30 may comprises an inertial sensor for measuring acceleration of said body part 42 in at least one spatial dimension, or in some embodiments all three spatial dimensions. This inertial sensor measures the motion of said body part 42 of the person 40 over time to generate an acceleration-over time signal that records the occurring accelerations at the wrist of the person 40 to which the portable device 10 may be attached. The motion sensor 30 may be used to switch on/off the device. The device may be automatically switched on upon receipt of an acceleration signal indicating that a user 40 employs the device. The device may be automatically switched off in case no acceleration signal is anymore generated and after a certain time period, such as 5, 8, 10, 12, 15, or 20 minutes has been passed to ensure that a user has in fact terminated in using the device. In addition, the signals of the motion sensor 30 may be employed by the processing unit to correct the measured heart rate 26 of the user and to obtain a more accurate heart rate signal 22. The vibration unit 32 may be also employed to indicate different events in real-time to the user, such as exceeding or falling below the defined single heart rate range 28, 30, or any alert. The user may assign specific signal sequences to different of the before mentioned events to allow proper distinction. As indicated above, also acoustic signals may be employed. It has been, however, found that such signals distract the user to a higher degree than vibration signals.

The system shown in FIG. 7 indicates the at least one of the first, second and third time periods as well as the monitored heart rate 26 of the user. The system may also comprise the vibration unit 32 which may be activated in case the current heart rate 26 passes the lower or upper limits of the defined single heart rate range 28, 30. The vibration unit 32 may be programmed to output different vibration signal sequences in response to a heart rate signal 22 falling below the defined single heart rate range 28 or exceeding the defined single heart rate range 30. The vibration unit 32 may emit signal sequences similar to Morse Codes, i.e. combination of short and/or long signal durations interrupted by breaks of variable times. In this manner the user may distinguish if the defined single heart rate range 28, 30 has fallen below or exceeded permitting in turn adapting of the exercise. In case the user 40 walks or runs, the vibration signal may indicate him/her to run faster or slower. In addition, the vibration unit 32 may be controlled in a manner that indication of a current heart rate 26 below and/or above the defined single heart rate range 28, 30 is performed after a certain threshold of e.g. 5, 10, 15, 20, 30, or 60 seconds. This assists in rendering the use of the system less annoying upon use of an inexperienced user who may easily have a current heart rate 26 below and/or above the defined single heart rate range 28, 30. In addition, the vibration sensor may give a signal to the user upon passing of preset time period indicating to switch from an exercise program to a program designed for training the physical activity, or vice versa. In addition, the vibration unit 32 may signal that a preset training time has been passed. It will be readily understood, that all above mentioned possibilities may be indicated with individual signal sequences which may be defined by the user. The system shown in FIG. 7 further shows the motion signal 24 that is received form the motion sensor 30 and used to trigger the start and/or stop of the measurement.

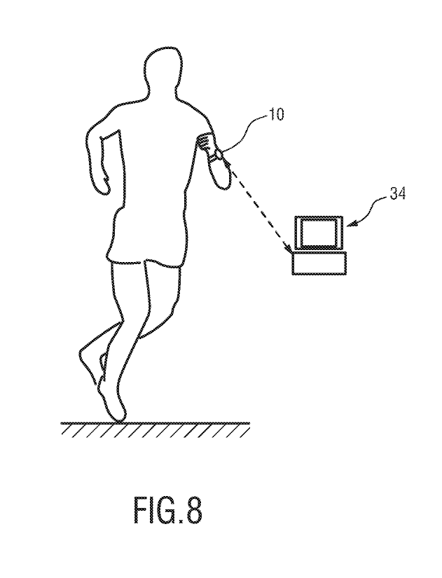

FIG. 8 shows another schematic appliance of the system wherein the first, second and third time periods T₁, T₂, and T₃ are indicated to a remote device 34. As noted above, this example is not limited to the processing and indication of three time periods. Processing and indication of one or two time periods is encompassed as well. Said remote device 32 may be a remote control unit, such as a watch or a mobile device having an appropriate app or a remote computer or laptop, which may be supervised by a medical practitioner who may in turn inform/instruct the current user of the system upon occurrence of any abnormality in the values indicated. Alternatively, the remote control unit serves to input and output training results. It is clear that a data connection between the remote device 34 and the device 10 may be realized in a variety of ways, e.g. using near field communication techniques, an Internet connection or radio communication techniques. The use of a remote control unit may be used in case the portable device is attached to a part of the person 40 difficult to see, such as chest, leg, around the neck, at or in an ear.

While the various embodiments has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing principles, methods, and systems described herein, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. System for monitoring heart rate of a user comprising:
an input unit for defining a single heart rate range;
a time measurement unit;
a heart rate sensor for measuring the heart rate of the user;
a processing unit for determining a first time period T₁ the measured heart rate is below the defined single heart rate range, a second time period T₂ the measured heart rate is inside the defined single heart rate range and a third time period T₃ the measured heart rate is above the defined single heart rate range; and
an output unit for indicating at least one of the first time period T₁, the second time period T₂ and the third time period T_{3.}

2. System according to claim 1, wherein the output unit is configured to simultaneously indicate the first time period T₁, the second time period T₂ and the third time period T_{3.}

3. System according to claim 1, wherein the system is at least partly arranged in a wrist-worn device or wherein the system is at least partly arranged in an in-ear device.

4. System according to claim 1, wherein the heart rate sensor is arranged remotely from the input unit, the time measurement unit, the processing unit, and the output unit.

5. System according to claim 1, wherein the heart rate sensor is part of a chest strap.

6. System according to claim 1, wherein the input unit is a mechanical or electronic button.

7. System according to claim 1, wherein the output unit comprises visual, acoustic and/or haptic feedback devices.

8. System according to claim 1, wherein the output unit comprises an acoustic signaling unit for acoustically indicating the at least one of the first time period T₁, the second time period T₂ and the third time period T_{3.}

9. System according to claim 1, wherein the output unit shows the current heart rate.

10. System according to claim 1, wherein the input unit for defining the single heart rate range is arranged remotely from the heart rate sensor.

11. System according to claim 1, wherein the input unit comprises a button configured to enter a perceived exertion of an exercise or an activity.

12. System according to claim 1, wherein the system comprises a vibration unit for indicating that the current heart rate is below and/or above the defined single heart rate range.

13. System according to claim 1, further comprising a motion sensor for activating the system upon receipt of a motion signal.

14. Method for monitoring the heart rate of a user, comprising:
defining a single heart rate range;
measuring the heart rate of the user over time to generate a heart rate signal;
determining a first time period T₁ the measured heart rate is below the defined single heart rate range, a second time period T₂ the measured heart rate is inside the defined single heart rate range and a third time period T₃ the measured heart rate is above the defined single heart rate range; and
indicating at least one of the first time period T₁, the second time period T₂ and the third time period T₃.

15. Method according to claim 14, wherein the at least one of the first, second and third time periods are indicated visually and/or acoustically.
